(19) United States
(12) Patent Application Publication
(10) Pub. No.: US 2011/0206177 A1
(43) Pub. Date: Aug. 25, 2011

(54) RADIOGRAPHIC IMAGE GENERATING APPARATUS AND RADIOGRAPHIC IMAGE GENERATING METHOD

(75) Inventor: Koichi HIRASAWA, Ashigarakami-gun (JP)

(73) Assignee: FUJIFILM CORPORATION, Tokyo (JP)

(21) Appl. No.: 13/005,341

(22) Filed: Jan. 12, 2011

(30) Foreign Application Priority Data
Feb. 25, 2010 (JP) 2010-040899

(51) Int. Cl. A61B 6/03

(52) U.S. Cl. 378/16

(57) ABSTRACT

A radiographic tomography image generating apparatus includes an irradiated dose determining unit for determining an irradiation dose at each of respective irradiating positions (present position) in order that a reached dose at a reference position on a radiation detecting device is made constant, and a radiation controller for controlling a radiation irradiator to apply radiation depending on the irradiating position (present position) and based on the irradiation dose determined by the irradiated dose determining unit.

### CROSS-REFERENCE TO RELATED APPLICATION

This application is based upon and claims the benefit of priority from Japanese Patent Application No. 2010-040899 filed on Feb. 25, 2010, of which the contents are incorporated herein by reference.

### BACKGROUND OF THE INVENTION

1. Field of the Invention

The present invention relates to a radiographic image generating apparatus and a radiographic image generating method for moving a radiation irradiator successively to a plurality of positions, applying radiation to a subject from the radiation irradiator at each of the positions over a radiation detector, acquiring a plurality of radiographic images output from the radiation detector, and rearranging the acquired radiographic images in order to generate a diagnostic image of the subject.

2. Description of the Related Art

In recent years, in order for X-ray image capturing apparatus to make a detailed observation of a local region of a subject, there has been proposed a tomosynthesis image capturing process in which a radiation irradiator, e.g., an X-ray tube, is moved over the subject while applying radiation to the subject from the radiation irradiator at different angles to capture radiographic images of the subject, and processing the captured radiographic images in order to generate a tomographic image, which is representative, with emphasis, of a desired sectional plane across the subject.

In such a tomosynthesis image capturing process, a plurality of radiographic images of the subject, which are captured at different angles, are acquired, and the acquired radiographic images are processed to reconstruct a tomographic image of the subject. The tomographic image can be generated by performing a given image processing technique on the radiographic images, and thereafter adding the processed radiographic images. According to one example of such a process for capturing a plurality of radiographic images at different angles, the radiation irradiator is moved along a circular track, as with a CT (Computerized Tomography) image capturing process.

Japanese Laid-Open Patent Publication No. 2008-062058 discloses a system for detecting in detail in a three-dimensional image shades of a tumor mass or a small calcified region, which has been extracted by a primary sampling process. More specifically, an angular distribution of a radiation dose, which is suitable for detecting the shades, is determined, and radiation doses are assigned from a total radiation dose to respective irradiation angles (see paragraph [0076], FIG. 5b). The process is effective to prevent the focal depth from being lowered in a region of interest, for thereby keeping the image sharp.

Japanese Laid-Open Patent Publication No. 2009-011639 discloses an apparatus for correcting pixel values of radiographic images, which are acquired at respective irradiation angles depending on the distance and angular relationship between a radiation irradiator and a radiation detector (see paragraphs [0064], [0074], FIG. 6). The disclosed apparatus can acquire radiographic images generated from radiation doses that are detected by the radiation detector, and which are converted into a substantially constant level, so that a radiographic image reconstructed from the radiographic images is free of density irregularities.

In a tomosynthesis image capturing process as well as in an elongate image capturing process, a radiation irradiator applies radiation of a constant dose to a radiation detector while the radiation detector moves successively to a plurality of positions. Therefore, each time that a radiographic image is captured, the distance between the radiation irradiator and the radiation detector, at the position to which the radiation detector has moved, may be subject to variations. Alternatively, the radiation detector may be fixed while the radiation irradiator moves successively to a plurality of positions. In such a modified system, however, the distance between the radiation irradiator and the radiation detector also may be subject to variations each time that a radiographic image is captured. As the distance becomes greater, the radiation dose applied to and transmitted through the subject is made smaller. Conversely, as the distance becomes smaller, the radiation dose applied to and transmitted through the subject is made greater. Stated otherwise, the radiation dose, which contributes to generation of each radiographic image, differs depending on the relative positional relationship between the radiation irradiator and the radiation detector.

When the radiation dose detected by the radiation detector is relatively small, the value of the image signal output from the radiation detector, which is representative of image information, is small, resulting in a small S/N (Signal to Noise) ratio in the image generating system. As a result, the image plotting capability in a region where radiation absorption contrast is low becomes reduced. This problem manifests itself particularly when the distance between the radiation irradiator and the radiation detector, at the position to which the radiation detector has moved, is large.

The system disclosed in Japanese Laid-Open Patent Publication No. 2008-062058 is designed so as to determine a distribution of radiation doses depending on the priority of a region of interest. The system is not aimed at increasing the image plotting capability for an entire diagnostic image.

The apparatus disclosed in Japanese Laid-Open Patent Publication No. 2009-011639 is designed in order to subsequently correct pixel values, so as to uniformize radiation dose irregularities between image areas of a radiographic image. The apparatus is not capable of increasing the S/N ratio as referred to above. Thus, the disclosed apparatus cannot increase the image plotting capability in a region where radiation absorption contrast is low.

### SUMMARY OF THE INVENTION

An object of the present invention is to provide a radiographic image generating apparatus and a radiographic image generating method, which are capable of preventing the image plotting capability from becoming lowered in a region where radiation absorption contrast is low, even when a radiation irradiator, which is disposed in confronting relation to a radiation detector, is moved successively to a plurality of positions.

According to the present invention, there is provided a radiographic tomography image generating apparatus comprising a radiation image acquiring assembly for acquiring a plurality of radiographic images output from a radiation detecting device, the radiation image acquiring assembly including a moving mechanism for moving a radiation irradiator successively to a plurality of positions and a radiation controller for controlling the radiation irradiator to apply radiation to a subject over the radiation detecting device at each irradiating position making up the plurality of positions while the radiation irradiator is moved by the moving mechanism, an image reconstructor for processing the radiographic images acquired by the radiation image acquiring assembly in order to generate a diagnostic image of the subject, and an irradiated dose determining unit for determining an irradiation dose at each irradiating position in order that a reached dose at a reference position on the radiation detecting device is made constant, wherein the radiation controller controls the radiation irradiator in order to apply radiation depending on the irradiating position and based on the irradiation dose as determined by the irradiated dose determining unit.

Inasmuch as the irradiated dose determining unit is provided for determining an irradiation dose at each irradiating position in order that a reached dose at a reference position on the radiation detecting device is made constant, the reached dose of radiation applied from each irradiating position is substantially constant, regardless of the distance between the radiation irradiator and the radiation detector, and the S/N ratio of the image generating apparatus also is substantially constant. Accordingly, when the radiation irradiator, which is disposed in confronting relation to the radiation detecting device, is moved to a plurality of positions, a reduction in the image plotting capability in a region where radiation absorption contrast is low can be prevented.

Preferably, the image generating apparatus further comprises a storage unit for storing irradiating information that associates the irradiating position with the irradiation dose, wherein the irradiated dose determining unit determines the irradiation dose depending on the irradiating position by referring to the irradiating information stored in the storage unit.

Preferably, the moving mechanism moves the radiation irradiator under a positional relationship, such that a distance between the radiation irradiator and the reference position varies.

Preferably, the moving mechanism moves the radiation irradiator on a prescribed straight track. Thus, compared with other tracks, the moving mechanism can be made simple, and radiographic images are prevented from becoming blurred (deteriorated in image quality) due to vibrations caused when the radiation irradiator is moved.

Preferably, the irradiating information comprises table data, which are successively arranged depending on the irradiating position, and the irradiated dose determining unit determines the irradiation dose by successively reading the table data at a prescribed timing depending on movement of the radiation irradiator. Thus, a radiation dose depending on the irradiating position can be determined without the need for acquiring positional information of the radiation irradiator each time radiographic images are taken.

Preferably, the image generating apparatus further comprises a setting unit for setting a type of radiation detecting device, wherein the storage unit stores the table data depending on the type of radiation detecting device.

Preferably, the image generating apparatus further comprises a setting unit for setting a size of the subject, wherein the storage unit stores the table data depending on the size of the subject.

Preferably, the table data comprise data defined by an mAs value of the radiation.

Preferably, the table data comprise data defined by a tube current supplied to the radiation irradiator.

Preferably, the table data comprise data defined by an irradiation time of the radiation.

Preferably, the image generating apparatus performs a tomosynthesis image capturing process or an elongate image capturing process.

According to the present invention, there also is provided a radiographic image capturing method comprising the steps of moving a radiation irradiator successively to a plurality of positions, determining an irradiation dose at each irradiating position making up the plurality of positions in order that a reached dose at a reference position on a radiation detecting device is made constant, controlling a radiation irradiator to apply radiation at the determined irradiation dose to a subject over the radiation detecting device, and acquiring a plurality of radiographic images output from the radiation detecting device and processing the radiographic images to generate a diagnostic image of the subject.

With the radiographic image capturing apparatus and the radiographic image capturing method according to the present invention, since an irradiation dose at each irradiating position is determined in order that the reached dose in the reference position on the radiation detecting device is made constant, the reached dose of the radiation applied from each irradiating position is substantially constant, regardless of the distance between the radiation irradiator and the radiation detector, and the S/N ratio of the image generating apparatus also is substantially constant. Accordingly, when the radiation irradiator, which is disposed in confronting relation to the radiation detecting device, is moved to a plurality of positions, a reduction in the image plotting capability in a region where radiation absorption contrast is low can be prevented.

The above and other objects, features, and advantages of the present invention will become more apparent from the following description when taken in conjunction with the accompanying drawings in which a preferred embodiment of the present invention is shown by way of illustrative example.

### BRIEF DESCRIPTION OF THE DRAWINGS

FIG. 1 is a schematic view, partially in block form, of a radiographic image generating apparatus according to an embodiment of the present invention;

FIG. 2 is a circuit diagram, partially in block form, of a radiation detecting device incorporated in the radiographic image generating apparatus shown in FIG. 1;

FIG. 3 is a diagram showing a first example illustrative of a positional relationship between a radiation irradiator, a subject, and the radiation detecting device;

FIG. 4 is a flowchart of an operation sequence of the radiographic image generating apparatus shown in FIG. 1;

FIG. 5 is a diagram showing a second example illustrative of a positional relationship between the radiation irradiator, the subject, and the radiation detecting device; and

FIG. 6 is a diagram showing a third example illustrative of a positional relationship between the radiation irradiator, the subject, and the radiation detecting device.

### DESCRIPTION OF THE PREFERRED EMBODIMENTS

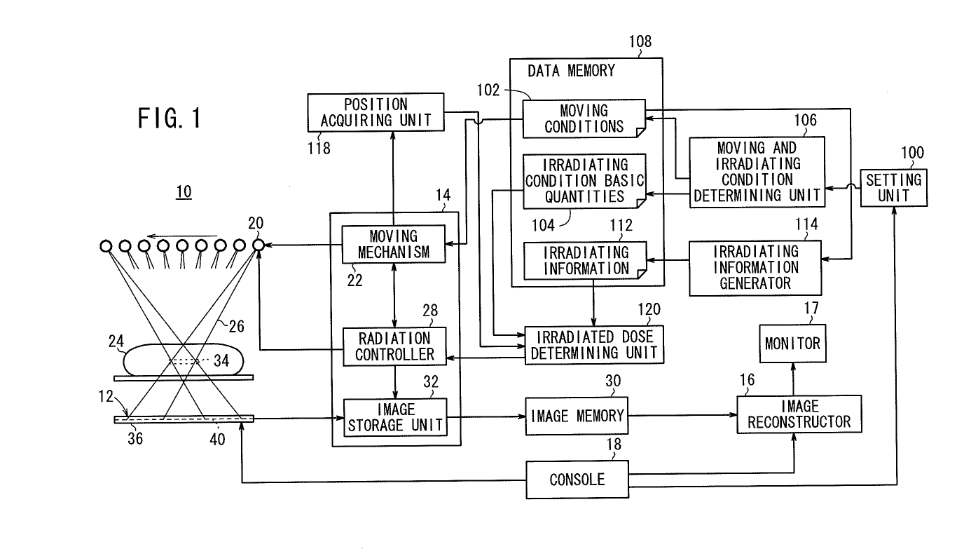

A radiographic image generating method according to an embodiment of the present invention, in relation to a radiographic image generating apparatus that carries out the radiographic image generating method, will be described in detail below with reference to FIGS. 1 through 6. Like or corresponding parts are denoted by like or corresponding reference characters throughout the views.

As shown in FIG. 1, an image generating apparatus 10, which constitutes a radiographic image generating apparatus according to an embodiment of the present invention, includes a radiation detecting device 12, a radiographic image acquiring assembly 14, an image reconstructor 16, a monitor 17, and a console (controller) 18 for controlling the radiation detecting device 12, the radiographic image acquiring assembly 14, the image reconstructor 16, and the monitor 17.

The radiographic image acquiring assembly 14 includes a radiation irradiator 20 disposed in confronting relation to the radiation detecting device 12, a moving mechanism 22 for moving the radiation irradiator 20 successively to a plurality of preset positions, a radiation controller 28 for controlling the radiation irradiator 20 to apply radiation 26 to a subject 24 over the radiation detecting device 12 when the radiation irradiator 20 reaches each of preset positions, each of which make up a prescribed position (irradiating position), and an image storage unit 32 for successively storing radiographic images, which are sent from the radiation detecting device 12, in an image memory 30 in chronological order, for example. The radiographic image acquiring assembly 14 operates to acquire a plurality of radiographic images from the radiation detecting device 12 when the radiation irradiator 20 applies radiation 26 to the subject 24 over the radiation detecting device 12 in different directions, and while the radiation irradiator 20, which is disposed in confronting relation to the radiation detecting device 12, moves successively to the preset positions. In the embodiment shown in FIG. 1, the moving mechanism 22 moves the radiation irradiator 20 along a straight track while the radiation detecting device 12 is fixed in position. However, the radiation irradiator 20 and the radiation detecting device 12, which are disposed one on each side of the subject 24, may be moved synchronously in mutually opposite horizontal directions.

The radiographic image acquiring assembly 14 operates in two modes, i.e., a mode for capturing an individual radiographic image each time that the radiation irradiator 20 reaches one of the preset positions, and a mode for capturing an image as a combination of individual radiographic images captured when the radiation irradiator 20 reaches the respective preset positions. The former mode will hereinafter be referred to as a “radiographic image capturing mode” and the latter mode as a “tomosynthesis image capturing mode.”

The image reconstructor 16 processes radiographic images stored in the image memory 30 in order to generate or reconstruct a tomographic image of the subject 24, i.e., a tomographic image (diagnostic tomographic image) of a region 34 of interest of the subject 24 arranged parallel to a detecting surface of the radiation detecting device 12. The image reconstructor 16 may reconstruct a tomographic image according to a reconstructing process, such as a simple back-projection process or a filtered back-projection process, for example. A simple back-projection process is a process for back-projecting a plurality of radiographic images without applying a reconstruction filter, and then adding the radiographic images into a reconstructed image. There are two types of filtered back-projection processes, i.e., a process for applying a reconstruction filter as a convolution filter to a plurality of radiographic images, back-projecting the radiographic images, and then adding the radiographic images into a reconstructed image, and a process for Fourier-transforming a plurality of radiographic images into frequency-domain data, applying a reconstruction filter to the frequency-domain data, back-projecting the frequency-domain data, and thereafter adding the radiographic images into a reconstructed image. Either of such filtered back-projection processes may be employed. The simple back-projection process and the filtered back-projection process will collectively be referred to as a “back-projection process.”

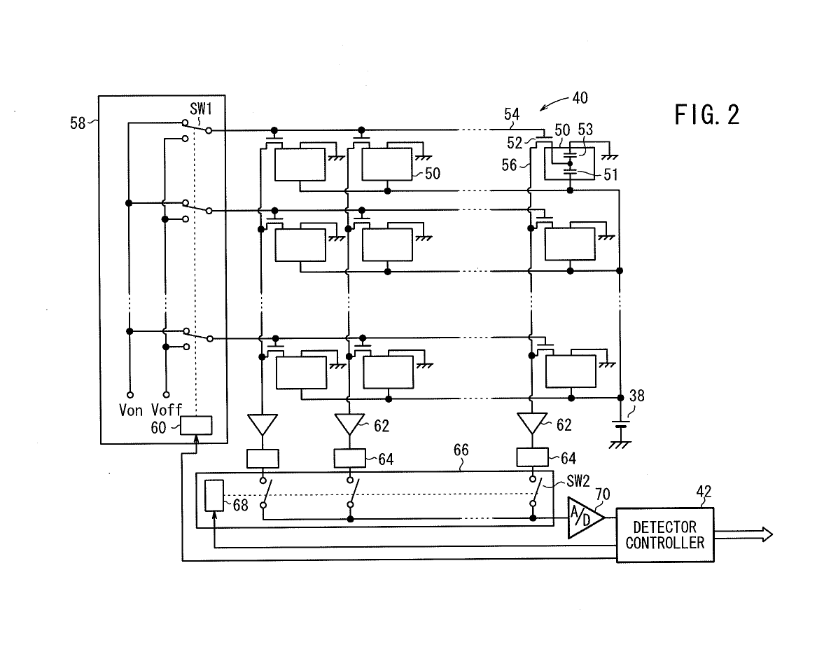

The radiation detecting device 12 includes a casing 36, a battery 38 (see FIG. 2) housed in the casing 36, a radiation detector 40 housed in the casing 36, and a detector controller 42 housed in the casing 36.

As shown in FIG. 2, the radiation detector 40 comprises an array of thin-film transistors (TFTs) 52 arranged in rows and columns, a photoelectric conversion layer 51 made of a material such as amorphous selenium (a-Se) for generating electric charges upon detection of radiation 26, the photoelectric conversion layer 51 being disposed on the array of TFTs 52, and an array of storage capacitors 53 connected to the photoelectric conversion layer 51. When radiation 26 is applied to the radiation detector 40, the photoelectric conversion layer 51 generates electric charges, and the storage capacitors 53 store the generated electric charges. Then, the TFTs 52 are turned on one row at a time to read the electric charges from the storage capacitors 53 as an image signal. In FIG. 2, the photoelectric conversion layer 51 and one of the storage capacitors 53 are shown as constituting a pixel 50, wherein the pixel 50 is connected to one of the TFTs 52. Details of the other pixels 50 are omitted from illustration. Since the structure of amorphous selenium tends to change and functions thereof are lost at high temperatures, amorphous selenium needs to be used within a certain temperature range. Therefore, some means for cooling the radiation detector 40 should preferably be provided in the casing 36.

The TFTs 52 connected to the respective pixels 50 are connected to respective gate lines 54 extending parallel to the rows, and to respective signal lines 56 extending parallel to the columns. The gate lines 54 are connected to a line scanning driver 58, and the signal lines 56 are connected to a multiplexer 66 that serves as a reading circuit.

The gate lines 54 are supplied with control signals Von, Voff for turning on and off the TFTs 52 along the rows from the line scanning driver 58. The line scanning driver 58 comprises a plurality of first switches SW1 for switching between the gate lines 54, and an address decoder 60 for outputting a selection signal for selecting one of the first switches SW1 at a time. The address decoder 60 is supplied with an address signal from the detector controller 42.

The signal lines 56 are supplied with electric charges stored in the storage capacitors 53 of the pixels 50 through the TFTs 52, which are arranged in columns. The electric charges supplied to the signal lines 56 are amplified by amplifiers 62 connected respectively to the signal lines 56. The amplifiers 62 are connected through respective sample and hold circuits 64 to the multiplexer 66. The multiplexer 66 comprises a plurality of second switches SW2 for successively switching between the signal lines 56, and an address decoder 68 for outputting a selection signal for selecting one of the second switches SW2 at a time. The address decoder 68 is supplied with an address signal from the detector controller 42. The multiplexer 66 has an output terminal connected to an A/D converter 70. Radiographic image signals generated by the multiplexer 66 based on electric charges from the sample and hold circuits 64 are converted by the A/D converter 70 into digital image signals representing radiographic image information, which are supplied to the detector controller 42. The detector controller 42 supplies the digital image signals to the image memory 30 (see FIG. 1), which stores the supplied digital image signals. In summary, each time that the first image generating apparatus 10 operates in a radiographic image capturing mode, the radiation detecting device 12 outputs a radiographic image. Radiographic images, which are successively output from the radiation detecting device 12, are stored in the image memory 30 in chronological order, for example.

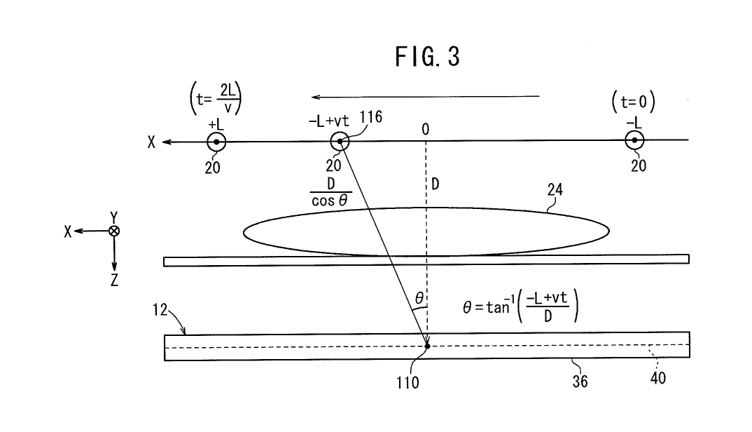

As shown in FIG. 1, the image generating apparatus 10 also includes a setting unit 100 for setting image capturing conditions based on an instruction from the operator who operates the console 18, a moving and irradiating condition determining unit 106 for determining moving conditions 102 and irradiating condition basic quantities 104 with respect to the radiation irradiator 20 based on image capturing conditions supplied from the setting unit 100, a data memory (storage unit) 108 for storing the moving conditions 102 and the irradiating condition basic quantities 104, etc., determined by the moving and irradiating condition determining unit 106, an irradiating information generator 114 for generating irradiating information 112 for keeping the dose (hereinafter referred to as a “reached dose”) of the radiation 26 constant at a reference position 110 (see the first example shown in FIG. 3) in the radiation detecting device 12, a position acquiring unit 118 for acquiring a present position (irradiating position, see FIG. 3) 116 of the radiation irradiator 20 from the moving mechanism 22, and an irradiated dose determining unit 120 for determining the dose (hereinafter referred to as an “irradiated dose”) of radiation 26 to be irradiated from the radiation irradiator 20 based on the present position supplied from the position acquiring unit 118.

The moving conditions 102 and the irradiating condition basic quantities 104 will be described below with reference to FIG. 3. In the first example shown in FIG. 3, the radiation irradiator 20 moves at a constant speed v along a straight track, i.e., along the X-axis direction, parallel to the detecting surface of the radiation detecting device 12. More specifically, the radiation irradiator 20 starts the radiographic image capturing process at a time t=0 from a position X=−L, and ends the radiographic image capturing process at a time t=(2 L/v) upon reaching a position X=+L. The radiation irradiator 20 is spaced a distance D from the plane of the radiation detector 40, at a reference position 110.

The moving conditions 102 represent various variables for specifying the relative positional relationship between the radiation detecting device 12 and the radiation irradiator 20. Parameters concerning movement control of the radiation irradiator 20 include the direction in which the radiation irradiator 20 moves (i.e., in a positive X-axis direction, as in the first example shown in FIG. 3), the speed (v in the first example shown in FIG. 3) at which the radiation irradiator 20 moves, and other variables for specifying the shape of the track (i.e., in the first example shown in FIG. 3, a straight line which forms an angle of 0° with respect to the X-axis, a start position −L, and an end position +L). If during the radiographic image capturing process, the radiation detecting device 12 is moved or turned in synchronism with the radiation irradiator 20, the parameters concerning movement control of the radiation irradiator 20 further include various parameters for identifying movement of the radiation detecting device 12.

The irradiating condition basic quantities 104 represent irradiating condition parameters of the radiation 26 (see FIG. 1) at a time when the radiation irradiator 20 is positioned at an origin O. For example, such irradiating condition parameters include an irradiation dose (mAs value), a tube current (mA), an irradiation time (s), etc., of the radiation irradiator 20. When a series of radiographic images are captured successively, the irradiating condition parameters may further include conditions the settings of which are not changed, e.g., a tube voltage (kV), a filter type, the distance from a given position (D in the first example shown in FIG. 3), etc.

The image generating apparatus 10 according to the present embodiment basically is constructed as described above. Operations of the image generating apparatus 10 will be described below.

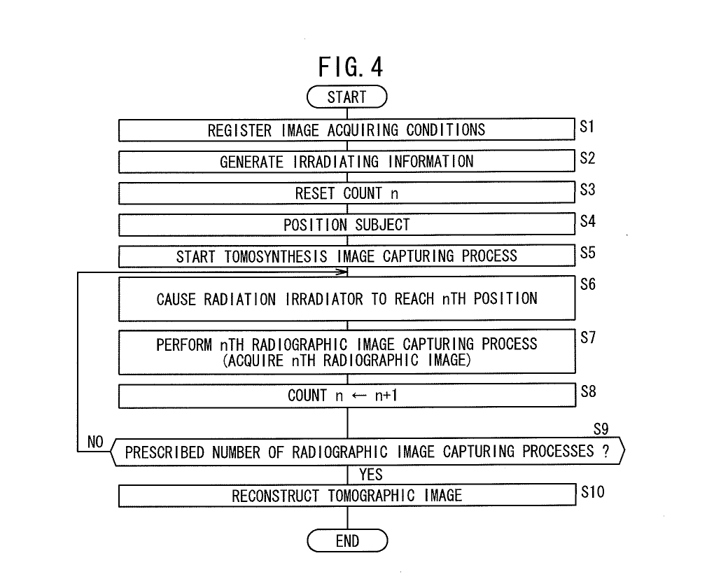

FIG. 4 is a flowchart of an operation sequence of the image generating apparatus 10 for obtaining an appropriate diagnostic image. The operation sequence of the image generating apparatus 10 will be described below, primarily with reference to FIGS. 1 and 4.

In step S1, patient information of a patient, i.e., the subject 24 to be imaged, is registered in the console 18 prior to capturing of radiographic images. If a body region to be imaged and an image capturing method have already been determined, such image capturing conditions are registered in advance in the console 18. Other image conditions, including the type of conversion process performed by the radiation detector 40, the size of the radiation detector 40, and the size of the subject 24, i.e., height, chest measurement, etc., may also be registered in advance in the console 18.

The image capturing conditions then are set from the console 18 into the setting unit 100, which supplies the image capturing conditions to the moving and irradiating condition determining unit 106. The moving and irradiating condition determining unit 106 determines moving conditions 102, along with irradiating condition basic quantities 104. The moving conditions 102 and the irradiating condition basic quantities 104, which have been determined, are stored in the data memory 108.

According to the present embodiment, the direction in which the radiation irradiator 20 moves (i.e., along a straight track in the positive X-axis direction), the speed (v) at which the radiation irradiator 20 moves, the start position (−L), and the end position (+L) are determined in order to define the moving conditions 102. The irradiation dose (mAs value), the tube current (mA), the irradiation time (s), the tube voltage (kV), the filter type, and the distance D when the radiation irradiator 20 is positioned at the origin O are determined in order to define the irradiating condition basic quantities 104.

Thereafter, in step S2, irradiating information 112 is generated. More specifically, the moving conditions 102, which are stored in the data memory 108, are supplied to the irradiating information generator 114, which generates irradiating information 112. The irradiating information 112 may be of any type, insofar as the irradiating information 112 associates respective irradiating positions of the radiation irradiator 20 with irradiation doses of radiation 26, which are applied at respective irradiating positions. For example, the irradiating information 112 may be in the form of table data, or may be defined as a conversion formula including a function format and coefficient data.

Alternatively, irradiating information 112 suitable for various image capturing conditions may be stored in the data memory 108, and optimum irradiating information 112 may be selected from the data memory 108 depending on image capturing conditions set in the setting unit 100.

According to a first example, the data memory 108 is capable of storing a plurality of items of irradiating information 112 depending on the conversion type of the radiation detector 40 that makes up the radiation detecting device 12. For example, the radiation detector 40 of the radiation detecting device 12 may be a direct conversion type, which directly converts the dose of applied radiation 26 into electric signals with the photoelectric conversion layer 51, an indirect conversion type including a scintillator for converting the applied radiation 26 into visible light and a solid-state detecting device made of amorphous silicon (a-Si) or the like for converting the visible light into electric signals (see Japanese Patent No. 3494683), or the radiation detector 40 may comprise an IP (Imaging Plate) made from a stimulable phosphor. Different image generating apparatus 10 may have respective irradiating information 112 inherent thereto, in view of different individual characteristics, even though such image generating apparatus 10 may include radiation detecting devices 12 of the same type.

According to a second example, the data memory 108 may store a plurality of items of irradiating information 112 depending on the size (detection size) of the radiation detector 40. For example, the definition of data intervals may be shared between respective table data, and the amount of data may be varied depending on the length (2L in the first example, as shown in FIG. 3) along which the radiation irradiator 20 moves, thereby facilitating processing operations performed by the image generating apparatus 10.

According to a third example, the data memory 108 may store a plurality of items of irradiating information 112 depending on the height of the subject 24. For example, the definition of data intervals may be shared between respective table data, and the amount of data may be varied depending on the height of the subject 24, thereby facilitating processing operations performed by the image generating apparatus 10.

According to a fourth example, the data memory 108 may store a plurality of items of irradiating information 112 depending on a chest measurement of the subject 24. For example, the thickness (i.e., height along the Z-axis direction) of the subject 24 may be predicted based on information representative of the chest measurement of the subject 24, in order to adjust the irradiation dose. Patient information representative of the gender, age, etc., of the subject 24, as well as the subject's chest measurement, may also be referred to.

In step S3, the count n of a counter, not shown, is reset to 0.

In step S4, the patient, i.e., the subject 24, is guided to the image generating apparatus 10, and is positioned depending on the body region to be imaged.

After the subject 24 has been positioned, control proceeds to step S5, in which the image generating apparatus 10 starts a tomosynthesis image capturing process based on an instruction from the operator who operates the console 18.

In step S6, the radiographic image acquiring assembly 14 controls the moving mechanism 22 so as to enable the radiation irradiator 20 to reach an nth position.

When the radiation irradiator 20 reaches the nth position, control proceeds to step S7 in which the image generating apparatus 10 performs an nth radiographic image capturing process. A process for determining an irradiation dose at the nth position (each irradiating position) will be described below.

Positional information of the radiation irradiator 20 is acquired by the position acquiring unit 118. Such positional information may include position coordinates, an angle, or an elapsed time.

In order to acquire the position coordinates (the present position 116 shown in FIG. 3) of the radiation irradiator 20, an existing position sensor may be used. Similarly, in order to acquire the angle (θ shown in FIG. 3) of the radiation irradiator 20, an existing angle sensor may be used.

The position or angle of the radiation irradiator 20 may be estimated using the elapsed time t from the start of the radiographic image capturing process. More specifically, the present position 116 or the angle θ of the radiation irradiator 20 can be estimated using the elapsed time t, which is acquired from the moving mechanism 22 or the like, and the moving conditions 102 stored in the data memory 108.

Furthermore, without using the moving mechanism 22, the elapsed time t (positional information) can be acquired under a timing control using a timer (not shown).

The positional information, which is acquired by the position acquiring unit 118, along with the irradiating condition basic quantities 104 and the moving conditions 102 stored in the data memory 108, are supplied to the irradiated dose determining unit 120. The irradiated dose determining unit 120 determines a dose of radiation 26, which is suitable for the present position.

The irradiating condition basic quantities 104 include an irradiation dose A_{O} (=Iₒ×Tₒ) [mAs], a tube current Iₒ [mA], and an irradiation time Tₒ [s] at the position of the origin O (see FIG. 3), for example. The irradiating information 112 includes information concerning a corrective coefficient f(θ) for correcting the irradiation dose with respect to each irradiating position (the angle θ shown in FIG. 3).

In the first example shown in FIG. 3, it can be seen that the coordinate X and the angle θ in the present position 116 at time t are given by X=−L+vt and θ=tan⁻¹(−L+vt/D), respectively, according to geometric analysis.

Since the dose that is reached is inversely proportional to the square of the distance from the radiation irradiator 20 in the absence of the subject 24, the corrective coefficient f(θ) for correcting the irradiation dose is determined by f(θ)=1/cos²θ.

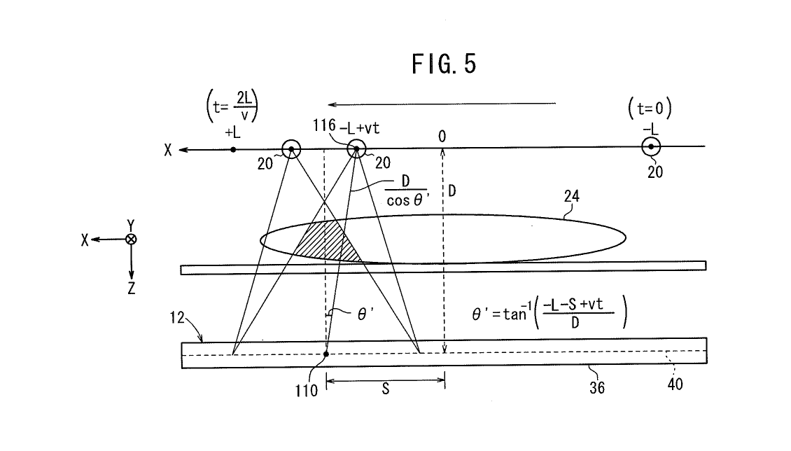

FIG. 5 shows a second example, which differs from the first example shown in FIG. 3, in that the coordinates of the reference position 110 are shifted by S in the X-axis direction. In the second example shown in FIG. 5, it can be seen that the coordinate X and the angle θ′ in the present position 116 at time t are given by X=−L+vt and θ′=tan⁻¹(−L−S+vt/D), respectively, according to geometric analysis. In this case, the corrective coefficient f(θ′) for correcting the irradiation dose is determined by f(θ′)=1/cos²θ′.

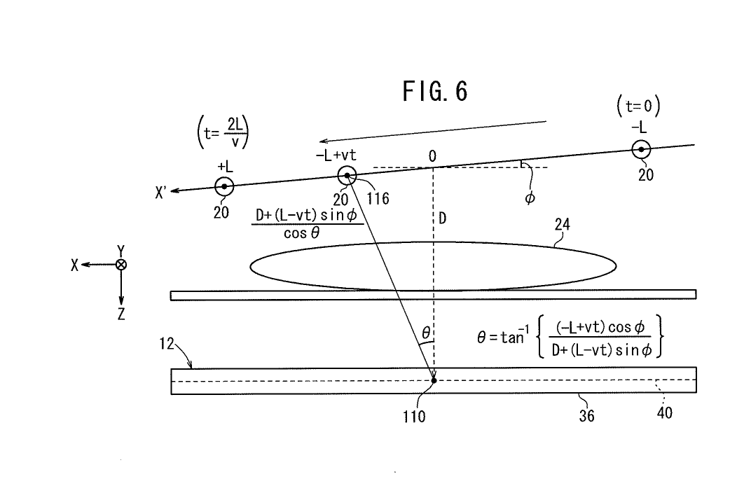

FIG. 6 shows a third example, which differs from the first example shown in FIG. 3, in that the straight track X′ of the radiation irradiator 20 is inclined with respect to the X-axis by φ. In the third example shown in FIG. 6, it can be seen that the coordinate X and the angle θ in the present position 116 at time t are given by X=(−L+vt)cos φ and θ=tan⁻¹[(−L+vt)cos φ/{D+(L−vt)sin φ}], respectively, according to geometric analysis. In this case, the corrective coefficient f(θ) for correcting the irradiation dose is determined by f(θ)={1+sin φ·(L−vt)/D}²/cos²θ.

The data format of the irradiating information 112 may be defined in various ways, insofar as the data format allows the appropriate dose to be determined depending on each irradiating position.

For example, data intervals of the table data should preferably be defined in association with positional information (position coordinates, angle, and elapsed time), which is acquired by the position acquiring unit 118. The amount of data does not matter, and data may be calculated between the table data by way of interpolation.

Data values of the table data may be defined as data (specifically, mAs values), which are directly representative of doses. In this case, the irradiated dose determining unit 120, which has acquired the irradiating information 112, can determine a tube current and an irradiation time as irradiating conditions based on the acquired irradiating information 112. If variables of the tube current and the irradiation time can assume discrete values only, then a combination of variables may be selected in order to make integrated values thereof closest to the acquired values.

For determining an mAs value, the tube current may be of a fixed value, and the irradiation time may be of a variable time. At this time, the irradiating information 112 may be defined as table data that is representative of a variable irradiation time.

Alternatively, for determining an mAs value, the tube current may be a variable value, and the irradiation time may be a fixed time. At this time, the irradiating information 112 may be defined as table data that is representative of a variable tube current.

The irradiating information 112, which is representative of a tube current and an irradiation time, may be stored separately in the data memory, and may be supplied simultaneously to the irradiated dose determining unit 120.

An nth radiographic image, which is produced in the nth radiographic image capturing process, is output from the radiation detecting device 12 and is stored chronologically in the image storage unit 32. Thereafter, the nth radiographic image is stored in the image memory 30. In this manner, the nth radiographic image is acquired in step S7.

Then, in step S8, the count n of the non-illustrated counter is incremented by +1.

Thereafter, in step S9, it is determined whether or not a prescribed number (e.g., 100) of radiographic image capturing processes have been performed, based on, for example, whether or not the count value n of the counter is greater than or equal to 100.

If a prescribed number of radiographic image capturing processes have not been performed, then control returns to step S6, and step S6 and steps subsequent thereto are carried out.

If it is determined that the prescribed number of radiographic image capturing processes have been performed in step S9, then control proceeds to step S10, in which the prescribed number of radiographic images stored in the image memory 30 are processed in order to reconstruct a tomographic image of the subject 24 according to a back-projection process. The generated tomographic image is displayed on the monitor 17, which is connected to the image generating apparatus 10.

Since, as described above, an irradiation dose at each irradiating position is determined in order to make the reached dose constant at the reference position 110 on the radiation detecting device 12, the reached dose of radiation 26 that is applied from each irradiating position is substantially constant, regardless of the distance between the radiation irradiator 20 and the radiation detector 40 (the reference position 110), while the S/N ratio of the image generating apparatus 10 remains substantially constant. Accordingly, when the radiation irradiator 20, which is disposed in confronting relation to the radiation detecting device 12, is moved to a plurality of positions, a reduction in the image plotting capability in a region where radiation absorption contrast is low can be prevented.

The present embodiment has been described mainly with respect to a tomosynthesis image capturing process. However, the present invention also is applicable to an elongate image capturing process, in which a body region longer than the radiation detector 40 is imaged, such as an image capturing process of the entire spine or an entire lower limb. The present invention further is applicable to an image capturing process in which the radiation irradiator 20 is not moved in position, but in which the orientation angle of the radiation irradiator 20 is varied in order to capture a plurality of radiographic images.

The present invention is not limited to the embodiment and various examples described above. Various changes and modifications may be made to the embodiment while remaining within the scope of the invention.

For example, a radiographic tomography image generating apparatus may employ a light readout radiation detector for acquiring radiographic image information. Such a light readout radiation detector operates in the following manner. When radiation is applied to a matrix of solid-state detecting devices, the solid-state detecting devices store an electrostatic latent image therein depending on the dose of applied radiation. For reading the stored electrostatic latent image, reading light is applied to the solid-state detecting devices, so as to cause the solid-state detecting devices to generate electric currents representative of the radiation image information. When erasing light is applied to the radiation detector, the radiographic image information, which is represented by a residual electrostatic latent image, is erased from the radiation detector, whereupon the radiation detector can be reused (see Japanese Laid-Open Patent Publication No. 2000-105297).

While the illustrated radiation detector 40 employs TFTs 52, the radiation detector may employ any of various other image capturing devices, such as a CMOS (Complementary Metal-Oxide Semiconductor) image sensor or a CCD (Charge-Coupled Device) image sensor, in which electric charges are shifted and transferred by shift pulses, which correspond to the gate signals used in the TFTs 52.

Although a certain preferred embodiment of the present invention has been shown and described in detail, it should be understood that various changes and modifications may be made to the embodiment without departing from the scope of the invention as set forth in the appended claims.

What is claimed is:

1. A radiographic tomography image generating apparatus comprising:
a radiographic image acquiring assembly for acquiring a plurality of radiographic images output from a radiation detecting device, the radiographic image acquiring assembly including a moving mechanism for moving a radiation irradiator successively to a plurality of positions and a radiation controller for controlling the radiation irradiator to apply radiation to a subject over the radiation detecting device at each irradiating position making up the plurality of positions while the radiation irradiator is moved by the moving mechanism;
an image reconstructor for processing the radiographic images acquired by the radiographic image acquiring assembly in order to generate a diagnostic image of the subject; and
an irradiated dose determining unit for determining an irradiation dose at each irradiating position in order that a reached dose at a reference position on the radiation detecting device is made constant,
wherein the radiation controller controls the radiation irradiator in order to apply radiation depending on the irradiating position and based on the irradiation dose as determined by the irradiated dose determining unit.

2. The image generating apparatus according to claim 1, further comprising:
a storage unit for storing irradiating information that associates the irradiating position with the irradiation dose,
wherein the irradiated dose determining unit determines the irradiation dose depending on the irradiating position by referring to the irradiating information stored in the storage unit.

3. The image generating apparatus according to claim 1, wherein the moving mechanism moves the radiation irradiator under a positional relationship, such that a distance between the radiation irradiator and the reference position varies.

4. The image generating apparatus according to claim 3, wherein the moving mechanism moves the radiation irradiator on a prescribed straight track.

5. The image generating apparatus according to claim 2, wherein the irradiating information comprises table data successively arranged depending on the irradiating position; and
the irradiated dose determining unit determines the irradiation dose by successively reading the table data at a prescribed timing depending on movement of the radiation irradiator.

6. The image generating apparatus according to claim 5, further comprising:
a setting unit for setting a type of the radiation detecting device,
wherein the storage unit stores the table data depending on the type of the radiation detecting device.

7. The image generating apparatus according to claim 5, further comprising:
a setting unit for setting a size of the subject,
wherein the storage unit stores the table data depending on the size of the subject.

8. The image generating apparatus according to claim 5, wherein the table data comprise data defined by an mAs value of the radiation.

9. The image generating apparatus according to claim 5, wherein the table data comprise data defined by a tube current supplied to the radiation irradiator.

10. The image generating apparatus according to claim 5, wherein the table data comprise data defined by an irradiation time of the radiation.

11. The image generating apparatus according to claim 2, wherein the moving mechanism moves the radiation irradiator under a positional relationship, such that a distance between the radiation irradiator and the reference position varies.

12. The image generating apparatus according to claim 11, wherein the moving mechanism moves the radiation irradiator on a prescribed straight track.

13. The image generating apparatus according to claim 1, wherein the image generating apparatus performs a tomosynthesis image capturing process or an elongate image capturing process.

14. A radiographic image capturing method comprising the steps of:
moving a radiation irradiator successively to a plurality of positions;
determining an irradiation dose at each irradiating position making up the plurality of positions in order that a reached dose at a reference position on a radiation detecting device is made constant;
controlling a radiation irradiator to apply radiation at the determined irradiation dose to a subject over the radiation detecting device; and
acquiring a plurality of radiographic images output from the radiation detecting device and processing the radiographic images to generate a diagnostic image of the subject.
